(19) United States
(12) Patent Application Publication
(10) Pub. No.: US 2022/0189584 A1
(43) Pub. Date: Jun. 16, 2022

(54) Microbiome Byproducts and Uses Thereof

(71) Applicant: PSOMAGEN INC., Rockville, MD (US)

(72) Inventors: Zachary Apte, San Francisco, CA (US); Jessica Richman, San Francisco, CA (US); Daniel Almonacid, San Francisco, CA (US); Valeria Marquez, San Francisco, CA (US)

(73) Assignee: PSOMAGEN INC., Rockville, MD (US)

(21) Appl. No.: 17/598,954

(22) PCT Filed: Mar. 27, 2020
(86) PCT No.: PCT/US2020/025284

§ 371 Date: Sep. 28, 2021

Related U.S. Application Data
(60) Provisional application No. 62/826,479, filed on Mar. 29, 2019.
(60) Provisional application No. 62/826,497, filed on Mar. 29, 2019.
(60) Provisional application No. 62/826,505, filed on Mar. 29, 2019.
(60) Provisional application No. 62/826,515, filed on Mar. 29, 2019.

(51) Int. Cl. G16B 40/20; G16B 15/20; A61K 35/741; G16B 20/00

(52) U.S. Cl. CPC G16B 40/20; G16B 15/20; G16B 30/10; G16B 20/00; A61K 35/741

(57) ABSTRACT

A method for treating a microorganism-related condition in a patient may include detecting microorganisms in a set of samples collected from a population and comparing a relative abundance of and co-occurrence between different microbial taxa in the set of samples. The method further includes associating a change in the relative abundance of or the co-occurrence between the microbial taxa with samples from people, among the population, with the microorganism-related condition and samples from people, among the population, without the microorganism-related condition to determine a target taxa. A blend of bacteriophages is then identified, the blend being configured to remove the target taxa from a community of microorganisms A therapeutic composition comprising the blend is then administered to the patient with the microorganism-related condition.

### CROSS-REFERENCE TO RELATED APPLICATIONS

The present application claims priority to U.S. Provisional Applications No. 62/826,479 filed on Mar. 29, 2019; 62/826,497 filed on Mar. 29, 2019; 62/826,505 filed on Mar. 29, 2019; and 62/826,515 filed on Mar. 29, 2019; each of which is incorporated herein by reference in its entirety for all purposes.

### BACKGROUND

Some antibacterial compounds produced by the human microbiota are involved in different biological functions associated to human health and/or disease conditions. Among the most common antibacterial compounds are lantibiotics, bacteriocins and microcins.

Bacteriocins and lantibiotics are antimicrobial peptides or proteins (e.g., −between 20 and 60 amino acids) synthesized by bacteria that inhibit or kill other microorganisms. Antibacterial compounds can promote a bactericidal or bacteriostatic effect, inhibiting cell growth. Bacteriocins have been mainly used as safe food preservatives because they are easily digested by the human gastrointestinal tract. However, some bacteriocins and lantibiotics are used in health related applications. Subtilosin A from Bacillus subtilis show anti-viral and spermicidal activities. Nisin, which is produced by some Gram-positive bacteria including Lactococcus and Streptococcus species, has the ability to control many Gram-positive pathogens, such as Streptococcus pneumoniae, Enterococci and Clostridium difficile. Microcins are small peptides (less than 10 kDa) derived exclusively from Enterobacteriaceae and have a potent antibacterial activity against close-related bacteria that produce it. The action of microcin B17 on sensitive Escherichia coli cells leads to the arrest of DNA replication and, consequently, to the induction of the SOS response. Diverse applications of antibacterial compounds are studied because some of them are recognized as Generally Recognized as Safe (GRAS) compounds by the FDA. Thus, antibacterial compounds, such as bacteriocins, lantibiotics and microcins are promising targets for health care biotechnology and pharmaceutical applications.

### SUMMARY

In a first aspect, a method for treating a microorganism-related condition in a patient may include detecting microorganisms in a set of samples collected from a population and comparing a relative abundance of and co-occurrence between different microbial taxa in the set of samples. The method further includes associating a change in the relative abundance of or the co-occurrence between the microbial taxa with samples from people, among the population, with the microorganism-related condition and samples from people, among the population, without the microorganism-related condition to determine a target taxa. A blend of bacteriophages is identified, the blend being configured to remove the target taxa from a community of microorganisms. A therapeutic composition comprising the blend is administered to the patient with the microorganism-related condition.

In a second aspect, a method for treating a microorganism-related condition in a patient may include detecting microorganisms in a set of samples collected from a population and comparing a relative abundance of and co-occurrence between different microbial taxa in the 115 set of samples. The method may further include associating a change in the relative abundance of or the co-occurrence between the microbial taxa with samples from people, among the population, with the microorganism-related condition and samples from people, among the population, without the microorganism-related condition to determine a target taxa. A blend of therapeutic microorganisms is identified, the blend being configured to change an abundance of the target taxa in a community of microorganisms. A therapeutic composition comprising the blend is administered to the patient with the microorganism-related condition.

In a third aspect, a method for identifying new bacteria-produced antibacterial compounds includes generating a database of antibacterial compounds produced by bacteria by screening known antibacterial compounds-producing microorganisms and antibacterial compounds and identifying, by a processor, binding regions of the antibacterial compounds from the database that bind other microorganisms by comparing sequence alignment of curated antibacterial compounds with a sequence alignment of reference proteomes. New bacteria-produced antibacterial compounds are identified based on the identified peptide motifs.

In a fourth aspect, method of producing a therapeutic composition may include identifying a protein from bacteria that produce metabolites underlying a microorganism-related condition and identifying, by a processor, a first inhibitor for the identified protein and a second inhibitor of a. protein orthologous to the identified protein using virtual high-throughput screening, A therapeutic composition comprising one or both of the first inhibitor and the second inhibitor is produced.

### BRIEF DESCRIPTION OF DRAWINGS

FIG. 1 illustrates an example of a pipeline to detect new bacteria-produced antibacterial compounds in accordance with an embodiment of the present disclosure.

FIG. 2 illustrates an example of a pipeline to modify the antibacterial compounds in accordance with an embodiment of the present disclosure.

### DETAILED DESCRIPTION

The following description of the technology is not intended to be limited to the various embodiments described below but to enable persons skilled in the art to make and use the same.

In an aspect of the present disclosure, a method for identifying new bacteria-produced antibacterial compounds is disclosed. In another aspect, a method for modifying the antibacterial compounds to improve antibacterial activity.

Embodiments can include, use, and/or otherwise be associated with one or more of:
a) Salivaricin A (e.g., a bacteriocin produce by Streptococcus salivarius K12 has been studied to inhibit malodour-associated bacterial species such as Streptococcus anginosis T29, Eubacterium saburreum and Micromonas micros; etc.)
b) Ruminococcin A (e.g., produced by Ruminococcus gnavus and Clostridium nexile has been studied against C. perfringens and C. difficile, suggesting as therapeutic agent against these pathogens. These pathogens are associated to antibiotic associated diarrhoea, and sporadic diarrhoea in humans; etc.).
c) Bacteriocin staphylococcin 188 (e.g., has been studied against Newcastle disease virus, influenza virus; etc.)

Embodiments can include can include one or more antibacterial compounds (e.g., in therapeutic compositions; etc.) from microbiota (e.g., any suitable microorganism taxa; etc.) for inhibiting and/or killing pathogenic bacteria. Embodiments can include inhibiting or killing pathogenic bacteria using one or more antibacterial compounds (e.g., in therapeutic compositions; etc.) from microbiota (e.g., any suitable microorganism taxa; etc.). Embodiments of a method can include using one or more bioinformatics approaches (e.g., bioinformatics pipeline) to identify one or more antibacterial compounds in microbiota (e.g., for inhibiting and/or killing pathogenic bacteria, etc.). Embodiments of a method can include one or more approaches using structural biology to design new antibacterial compounds, such as based on existing ones.

In embodiments, the new natural and/or modified antibacterial compounds can be used as treatment for disease, and/or for health care biotechnology and pharmaceutical applications. Additionally or alternatively, embodiments can be used for one or more of: food preservation, producing active probiotic culture, treatment of infections, antibiotic resistance to conventional antibiotics, post-surgical control of infectious bacteria, and/or as potential anti-cancer agents.

First stage (and/or performable at any suitable time and frequency): This pipeline allows to find new bacteria-produced antibacterial compounds.

First (and/or performable at any suitable time and frequency), a screening of known antibacterial compounds-producing microorganisms and antibacterial compounds is performed to generate a database of antibacterial compounds produced by bacteria. All related information and/or any suitable combination of information can be used for the next steps_{;} including the name of the antibacterial, the microorganisms that produce it, the application, host site and/or target microorganisms that inhibit and/or kill.

Then (and/or performable at any suitable time and frequency), curated antibacterial compounds (e.g., lantibiotic, bacteriocin and/or microcin; etc.) database is used to search against reference proteomes (e.g., from Uniprot or NCBI databases; etc.) using different sequence alignment algorithms (e.g., BLAST. FASTA. Clustal, among others; etc.). The alignment can be used to identify peptide motifs that can be useful to predict the binding region of antibacterial compounds to other microorganisms, and/or finally to identify new bacteria-produced antibacterial compounds.

Second stage (and/or performable at any suitable time and frequency): This pipeline allows to modify the antibacterial compounds to improve the antimicrobial activity.

The second approach can include modifying antibacterial peptides that have a defined tridimensional structure and have a known particular target (e.g., obtained from a structural database, e.g. Protein Data Bank, Bactibase, BAGEL, among others; etc.). Based on that, and/or based on the identification of relevant peptide motifs from the first stage (and/or suitable step), a structural analysis is performed to identify whether those motifs are exposed to the solvent and, therefore, can interact with proteins from other microorganisms. This analysis can be performed using solvent-accessible surface area (SAS.-) and/or any suitable aspects.

Then and/or performable at any suitable time and frequency), a molecular docking (as control) and/or suitable experiment can be performed to model the atomic interaction between the antimicrobial peptide or motif and the target from a microorganism known to be inhibited by the action of the antibacterial peptide. Both molecules are considered rigid, that is, the bonds do not rotate and maintain the secondary structure. Taking this into account, new antimicrobial peptides can be designed. To do this, modifications on segments of amino acids of antibacterial peptide are performed to get new antibacterial peptides with a better antimicrobial activity. The modifications include mutating each position of peptides for the remaining 19 amino acids (but any suitable number of amino acids at any suitable positions can be modified). Subsequently (and/or performable at any suitable time and frequency), docking between modified peptides and the target is performed. Thus, the new antibacterial peptide can bind with high affinity to the target, and therefore, can improve their antimicrobial activity.

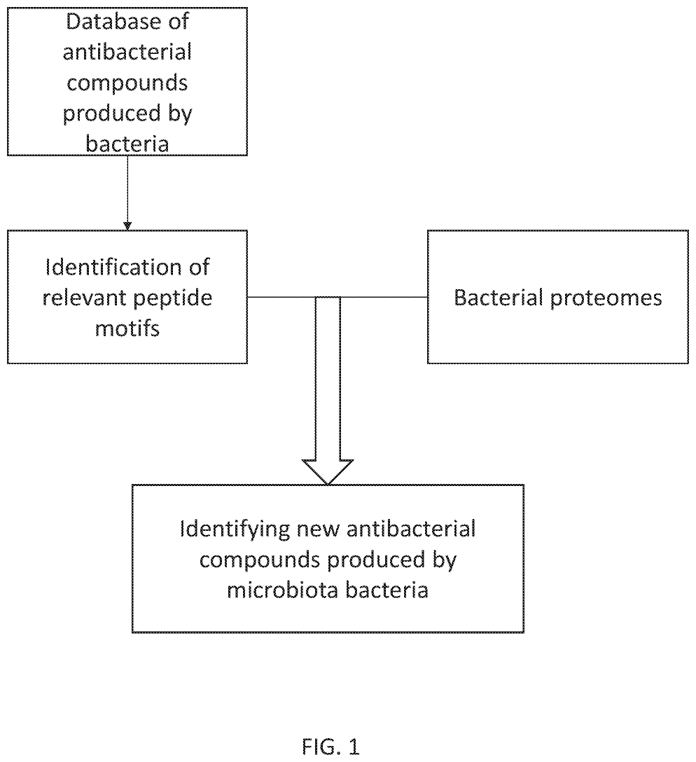

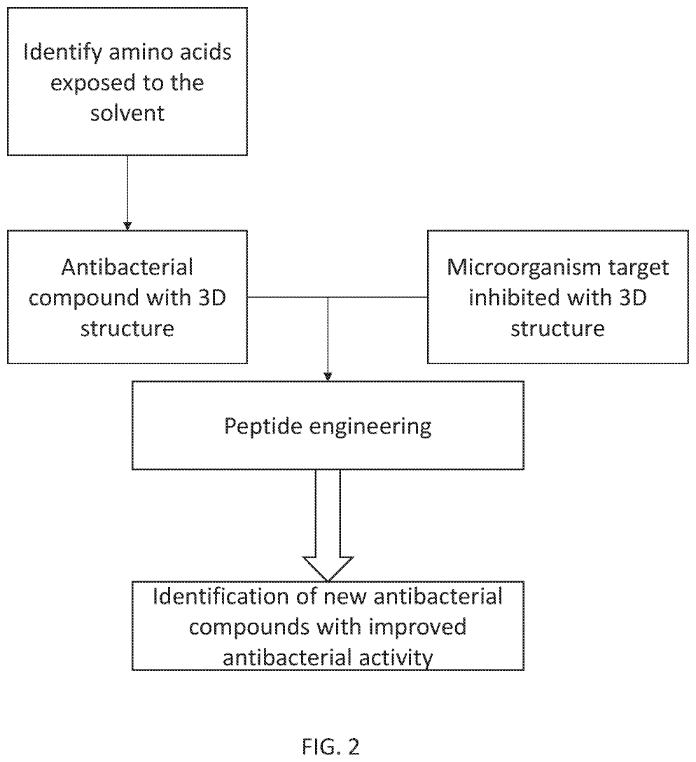

Embodiments can include a pipeline to identify new human bacteria-producing antibacterial compounds, a schematic of which is shown in FIG. 1. Embodiments can include a pipeline to modify the antibacterial compounds to get new ones, a schematic of which is shown in FIG. 2.

In another aspect of the present disclosure, a platform for selection of microorganisms for phage for treatment of conditions is disclosed.

Embodiments of a method can include detecting and/or otherwise determining) microorganisms (e.g., taxa) with increased abundances and/or that increase their abundances) in people with (e.g., associated with) a certain health condition of interest (e.g., microorganism-related condition; etc.). Embodiments of a method can include using one or more statistical approaches for comparing the relative abundance of the microbial taxa in a sample and associating the change in abundance (if any) between people with and/or without a certain health condition of interest, such as while considering the functions provided by the microorganisms to their human host, and/or the co-occurrence between different taxa. Embodiments of a method can include, based on this information (and/or suitable data described herein), a specific blend (e.g., combination) of one or more bacteriophages can be produced, applied and/or otherwise used to down-regulate the abundance of the target taxa, such as by removing the correlated taxa from the community, which can cause a potential (e.g., positive, etc.) effect over certain health condition(s) and/or other host properties.

Embodiments of a method can include identifying the change in relative abundance of microorganisms (e.g., as a consequence of one or more certain health conditions; etc.), and/or whose change is associated with the onset of the one or more health conditions. Embodiments can include generation and/or determination and/or can include therapeutic compositions including one or more custom bacteriophage blend combinations (e.g., prescription, etc.) for one or more users/patients, such as based on utilizing the data of their microbiome composition, compared with either (him/her)self in time window compositions, and/or compared with a reference population composition set.

Embodiments of a method can include Identifying which microorganisms increase their relative abundances (and/or have increase abundances) associated with a given health condition, showing a positive correlation. In specific examples, these taxa can be the target of a specific bacteriophage that can reduce their abundances, and/or remove them completely from the communities (e.g., user microbiome).

Examples for identifying increased taxa, such as associated with one or more conditions From a list of over 64000 Operational Taxonomic Units (OTUs), a subset was to be selected as positively associated with certain health conditions of interest.

An objective criteria can be defined for this selection. In specific examples, the criteria can include selecting a subset of samples collected , from users, who answered a comprehensive survey, specifically claiming they currently have the health condition of interest (and/or have been diagnosed with it, in case of chronic conditions, henceforth, the “condition group”). Additionally or alternatively, a subset of samples from users who specifically claimed not to have the condition of interest was selected (henceforth, the “control group”). However, any suitable criteria can be used (e.g., any suitable survey responses, etc.

The relative abundance of OTUs of these two cohorts was gathered, and statistically analyzed for detecting which microbial taxa are directly associated (i.e. its abundance is increased) in the condition group. In specific examples, two statistical approaches can be used but any suitable number and/or type of statistical approaches can be used. First, a logistic regression (with probit link and/or any suitable approach) is conducted on CLR-transformed relative data, using the condition of interest (i.e. ill vs healthy) as response variable, and OTUs abundance as predictors; but any suitable regression approach can be used. CLR transtbrmation was used to remove bias introduced in the data because of its relative nature (i.e. compositional data); but any suitable transformation approach can be used. Second, zero-inflated negative binomial regression was conducted for each OTU's relative abundance, with the condition of interest as predictor; but any suitable regression approach can be used. This analysis has the advantage that works well for severely left-skewed distributions, models separately zero and greater than zero abundances, and can perform better than Poisson regression in specific examples, because it is better at controlling for overdispersion in the data. Additionally, it works well on count data. Only OTUs that showed statistical difference in relative abundance P-value equal or less than 0.05; but any suitable threshold can be used) for both analyses were considered as potential candidates for removing them from the communities. Selected OTUs were then annotated to its corresponding taxonomic level using SILVA taxonomy. Output information includes information such as “regression coefficients”, which can be interpreted as the amount of change in relative abundance for each OTU estimated by the regression models under the condition of interest. A positive coefficient represents an increase in abundance, whereas a negative number represents a decrease in relative abundance.

Examples of combination of microorganisms to be included in a probiotic formulation for a specific condition:

Specific examples can include one or more therapeutic compositions including one or more new bacteriophage formulations (e.g., with any suitable amount of bacteriophages; etc.) as a treatment for one or more health conditions of interest, which can include any one or more phages capable of infecting the identified microorganisms. The origin of those bacteriophages may be from: natural sources, engineered sources (e.g. lysogenic viruses converted into lytic forms), synthetic production and/or any other method or source of origin. The delivery instrument of the bacteriophage blend/mixture can be: in liquid (e.g, syrup, saline solution, dairy products, etc.), solid (e.g. pills, food sources, etc.) and/or any other delivery instrument. The delivery mode can be oral, rectal, vaginal and/or any other mode of delivery.

In yet another aspect of the present disclosure, a platform for selection of microorganisms for a live biotherapeutic composition for treatment of certain microorganism-related conditions is disclosed.

Microbial communities inhabiting the human body provide their hosts with multiple beneficial functions, such as producing necessary molecules, improving the immune system, or preventing the colonization of harmful species. Over the past years, large amounts of scientific literature have described the association between some health conditions and the reduction or depletion of specific commensal microorganisms. It would be important (from a medical and commercial point of view) to replenish the microbial communities with its lost members in order to recover from, or ameliorate the symptoms of those health conditions.

Certain live microorganisms, when administered in adequate amounts, can provide different benefits to humans. These microorganisms, known as probiotics, have been used for many years. The most widely used probiotics are Saccharomyces, Lactobacillus and Bifidobacterium. However, the list of microorganisms suitable as probiotics Generally Regarded As Safe (GRAS) is expanding every day, thanks to the improvement of the technology for identifying microorganisms with more and more precision.

Organisms described by means of these new technologies are often called “next-generation probiotics” (NGPs), and can be used with very specific purposes, aiming to treat specific conditions. Because of this, they are also termed Live Biotherapeutics (LBPs).

Embodiments can include determination (e.g., identification, etc.) of, approaches associated with, suitable therapeutic compositions (e.g., live biotherapeutic compositions) including and/or any suitable method processes and/or system components including arid/or associated with microorganisms that show a decrease after antibiotics consumption and/or microorganisms with decreased abundance caused by any suitable factors (e.g., health conditions; behaviors; diet; etc.). Embodiments can include one or more such candidates for LBPs and/or suitable consumables (e.g., live biotherapeutics, probiotics, prehiotics, etc.) and/or therapeutic compositions.

Embodiments can include detecting microorganisms that reduce their abundances (and/or with reduced abundance) in people with one or more certain health conditions of interest (e.g., microorganism-related conditions; etc.). Embodiments can include applying statistical approaches that can compare the relative abundance of the microbial taxa in a sample and associate the chance in abundance (if any) between people with and without one or more certain health conditions of interest, such as considering the functions provided by the microorganisms to their human host, and/or the co-occurrence between different taxa. In embodiments, based on this information, there can be determination of, use of, and/or inclusion of a specific blend of LBPs and/or suitable consumables (e.g., live biotherapeutics, probiotics, prehiotics, etc. and/or therapeutics) and/or therapeutic compositions, such as can be produced to up-regulate the abundance of the target taxa, such as by repopulating the community with the depleted taxa.

Any suitable taxa described herein (and/or identifiable by approaches described herein) can be used in one or more LBPs and/or suitable consumables (e.g., live biotherapeutics, probiotics, prehiotics, etc.) and/or therapeutic compositions (e.g., therapeutics, etc.).

In a specific example, the method can include identifying microorganisms inhabiting the human gut (and/or suitable body site) that show a decrease after antibiotics consumption, which can become candidates for LBPs and/or suitable consumables (e.g., live biotherapeutics, probiotics, prehiotics, etc.) and/or therapeutic compositions.

Embodiments can include a method to identify the change in relative abundance of microorganisms as a consequence of (and/or otherwise associated with) a certain health condition, and/or whose change is associated with the onset of that health condition.

Embodiments can include consumables and/or other suitable therapeutic compositions including one or more combinations of microorganisms (e.g., described herein) that should be included in a potential LBP blend for the treatment of a certain health condition.

Embodiments can include identifying which microorganisms reduce their relative abundances (e.g., have reduced relative abundance) associated with a given health condition, such as for aiming to recovering the lost taxa and alleviating symptoms of health conditions produced as a consequence of the reduction of those taxa.

In specific examples, Section 1 (below) describes specific examples of method to identify bacterial taxa as described herein, such as to be included in a LBP formulation and/or suitable therapeutic compositions. Section 2 provides specific examples of the identified species.

1.1 Specific Examples of Method to Identify Bacteria that result Depleted after Antibiotic Consumption

From a list of over 64000 Operational Taxonomic Units (OTUs), a subset was to be selected as potential candidates for inclusion in a probiotic for recover the microbiota the onset of a disturbance (i.e. health condition, consumption of medication, etc). An objective criteria had to be defined for this selection. We opted for selecting a subset of samples from users, who answered a comprehensive survey, specifically claiming they currently have the health condition of interest (or have been diagnosed with it, in case of chronic conditions, henceforth, the “condition group”). Additionally, a subset of samples from users who specifically claimed not to have the condition of interest was selected (henceforth, the “control group”). However, any suitable criteria can be used to select different groups of users and/or samples. The relative abundance of OTUs of these two cohorts was gathered, and statistically analyzed for detecting which microbial taxa are inversely associated (i.e. its abundance is reduced) in the condition group. Two statistical approaches are to be used (but any suitable number and/or type of statistical approaches can be used). First, a logistic regression (with probit link) is conducted on CLR-transformed relative data, using the condition of interest (i.e. consumer vs non-consumer, ill vs healthy, etc) as response variable, and OTUs abundance as predictors. CLR transformation was used to remove bias introduced in the data because of its relative nature (i.e. compositional data). Second, zero-inflated negative binomial regression was conducted for each OTU's relative abundance, with the condition of interest as predictor. This analysis has the advantage that works well for severely left-skewed distributions, models separately zero and greater than zero abundances, and performs better than Poisson regression, because it is better at controlling for overdispersion in the data. Additionally, it works well on count data. Only OTUs that showed statistical difference in relative abundance (i.e. P-value equal or less than 0.05; but any suitable criteria conditions can be used) for both analyses were considered as potential candidates for inclusion in the probiotic. Selected OTUs were then annotated to its corresponding taxonomic level using SILVA taxonomy. Output information includes information such as “regression coefficients”, which can be interpreted as the amount of change in relative abundance for each OTU estimated by the regression models under the condition of interest. A negative coefficient represents a decrease in abundance, whereas a positive number represents an increase in relative abundance.

Functions provided by the bacterial community in the gut and/or suitable body sites are diverse, and usually redundant, meaning that more than only one taxon is involved in carrying out a certain function. Some conditions or host behaviors (e.g. consuming 115 antibiotics, medications or alcohol) introduce disturbances in the communities of microorganisms, which affects the abundances of the species inhabiting different locations of human body. As a consequence, some of the functions carried out by the microbiota are altered or even disappear. Therefore, a method of detecting which microbial taxa are reduced by the disturbances may also include the ecological services (i.e. metabolic functions) carried out by those taxa.

As an example, as shown in Table A taxa that showed different relative abundances in samples from people who consume (the condition group) and did not consume antibiotics (the control group). The table also shows the taxa that carry out the functions considered to be important to conserve after a course of antibiotics. The metabolic functions including pathogen inhibition, polysaccharides degradation, short chain fatty acids production, conjugated linoleic acid production and/or indole production, among others. For example, indole production improves barrier function and decrease intestinal inflammation in vitro and in vivo. Additionally, it decrease pathogen colonization.

All analyses were conducted in R statistical software. Pscl and MASS packages were used for the regression analyses. Compositions package was used for performing CLR transformation on data when necessary. However, any suitable statistical software and/or approaches and/or transformation software and/or approaches can be used.

1.2 Specific Examples of Method for Detecting Taxa Co-Occurrence.

The microbiota inhabiting different locations of the human body is structured as a biological community. Thus, it is expected that most of the taxa will show negative and positive interactions with others. Knowing the interactions between different taxa gives us more options to preserve or re-introduce some depleted taxa into the gut community following a disturbance. For example, if a taxon A is of interest, but it is not possible to add it to a probiotic, the mix a different taxon, B, which has a strong co-occurrence probability with taxon A, can be added. To gather this information about the positive interactions between taxa, a co-occurrence analysis is performed in a subset of samples of regarded as “control” (i.e. do not have the health condition or behaviour of interest), to know what are the patterns of positive interactions in a “normal” microbiota.

A threshold of 0.85 was set as the minimum probability of co-occurrence useful., but any suitable threshold can be used. As an example, a list of co-occurring taxa at genus level is provided, using as “control” group people who have not consumed antibiotics (Table B). The column “prob_cooccur” represents the probability of finding the two organisms in the sample sample, the column “p_gt” represents the probability that when one of the taxa is present, the other is also present. The “effects” column represent the effect size of the association between the taxa.

All analyses were conducted in R statistical software. Cooccur package was used for the co-occurrence analysis. However, any suitable statistical software and/or approaches and/or transformation software and/or approaches can be used.

2.1. Specific Examples of Combination of Microorganisms to be included in a Probiotic Formulation (and/or Suitable Therapeutic Composition) for one or more Specific Conditions.

Embodiments can include determination of and/or include one or more new LBP formulations (and/or suitable therapeutic compositions) as a treatment for the one or more conditions of interest, such as can include any one or more strains of the species detected to decrease in abundance (and/or be decreased in abundance) in samples from people with the condition.

2.1.1. Examples of Bacterial Species that resulted Depleted after Antibiotics Formulation.

In specific examples, in the following section, it will be described potential bacteria to be used as LBP and/or suitable consumables (e.g., live biotherapeutics, probiotics, prebiotics, etc.) and/or suitable therapeutic compositions.

In a first example, a new LBP formulation (and/or therapeutic composition) as an antibiotics recovery treatment can include at least one or more of the following strains and/or species: Enterococcus faecium, Lactobacillus rhamnosus, Lactobacillus salivarius, Bifidobacterium adolescentis, Bifidobacterium animal's, Lactobacillus gasseri, Bifidobacterium breve, Bifidobacterium catenulatum, Bifidobacterium pseudocatenulatum, Bifidobacterium stercoris, Lactobacillus reuteri, Lactobacillus fermentutn, Pediococcus pentosaceus, Lactobacillus helveticus, Lactobacillus brevis, Lactococcus lactis, Bacteroides xylanisolvens. The combination of all of them, or a subset of them, can be used for this treatment, diagnostics, and/or any suitable purpose. One or more of the described can include and/or be associated with all, or some of the following properties: pathogen inhibition, degradation of polysaccharides, degradation of mucin, short-chain fatty acids production, conjugation of linoleic acids production, production of GABA, indole production, modulation of immune response.

In a second example, a new LBP formulation (and/or therapeutic composition) as an antibiotics recovery treatment can include at least one or more strain and/or species: Faecalibacterium prausnitzii, Roseburia faecis, Roseburia hominis, Roseburia intestinalis, Anaerostipes caccae, Anaerostipes rhamnosivorans, Eubacterium limosurn, Eubacterium sp. ARC.2, Subdoligranulum variabile, Akkermansia muciniphila, Bifidobacterium adolescentis, Bifidobacterium animalis, Bifidobacterium breve, Bifidobacterium catenulatum, Bifidobacterium crudilactis, Bifidobacterium dentium, Bifidobacterium pseudocatenulatum, Bifidobacterium stercoris, Bifidobacterium thermacidophilum, Methanobrevibacter smithii, Roseburia sp. 499, Bacteroides dorei, Bacteroides massiliensis, Bacteroides plebeius, Bacteroides sp. 35AE37, Bacteroides thetaiotaomicron, Bacteroides xylanisolvens, Lactobacillus rhamnosus, Lactococcus lactis, Enterococcus faecium, Lactobacillus salivarius, Lactobacillus gasseri, Lactobacillus reuteri, Lactobacillus fermentum, Pediococcus pentosaceus, Lactobacillus helveticus. Lactobacillus brevis. One or more of such species have all, or some of the following properties: pathogen inhibition, degradation of polysaccharides, degradation of mucin, short-chain fatty acids production, conjugation of linoleic acids production, production of GABA, indole production, and/or modulation of immune response. Specific examples of the regression coefficient for each bacterial taxa, and some of their functions are described in table A.

TABLE A
Specific example of list of the taxa that showed to have different relative abundances between
antibiotic consumer and non-consumer subjects, along with the important functions these taxa perform.

| | | | | | Production of | | | | |
|---|---|---|---|---|---|---|---|---|---|
| | | | | | | Conjugated | | | |
| | Regression | Pathogen | Degradation of | | | linoleic | | | |
| Taxa | coefficient | inhibition | polysaccharides | mucin | SCFA | acid | Enterolactone | GABA | Indole |
| Faecalibacterium | −25.92 | | yes | | yes | | | | |
| prausnitzii | | | | | | | | | |
| Roseburia | −5.46 | | yes | | yes | | | | |
| faecis | | | | | | | | | |
| Roseburia | −5.19 | | yes | | yes | | | | |
| hominis | | | | | | | | | |
| Roseburia | −3.57 | | yes | | yes | | | | |
| intestinalis | | | | | | | | | |
| Anaerostipes | −0.98 | | | | yes | | | | |
| caccae | | | | | | | | | |
| Anaerostipes | −0.88 | | | | yes | | | | |
| rhamnosivorans | | | | | | | | | |
| Eubacterium | −0.41 | | | | yes | | | | |
| limosum | | | | | | | | | |
| Eubacterium | −0.41 | | | | | | yes | | |
| sp. ARC.2 | | | | | | | | | |
| Subdoligranulum | −0.40 | | | | yes | | | | |
| variabile | | | | | | | | | |
| Akkermansia | −0.16 | | | yes | yes | | | | |
| muciniphila | | | | | | | | | |
| Bifidobacterium | −0.17 | | | | yes | | | | |
| adolescentis | | | | | | | | | |
| Bifidobacterium | −0.16 | yes | | | | | | | |
| animalis | | | | | | | | | |
| Bifidobacterium | −0.15 | | yes | | | | | | |
| breve | | | | | | | | | |
| Bifidobacterium | −0.15 | | | | yes | | | | |
| catenulatum | | | | | | | | | |
| Bifidobacterium | −0.15 | | yes | | | | | | |
| crudilactis | | | | | | | | | |
| Bifidobacterium | −0.15 | | | | | | | yes | |
| dentium | | | | | | | | | |
| Bifidobacterium | −0.14 | | | | yes | | | | |
| pseudocatenulatum | | | | | | | | | |
| Bifidobacterium | −0.11 | | | | yes | | | | |
| stercoris | | | | | | | | | |
| Bifidobacterium | −0.11 | | | | | | | | |
| thermacidophilum | | | | | | | | | |
| Methanobrevibacter | −0.11 | | yes | | | | | | |
| smithii | | | | | | | | | |
| Roseburia sp. 499 | −0.06 | | yes | | | | | | |
| Bacteroides dorei | −0.06 | | yes | | | | | | yes |
| Bacteroides | −0.06 | | yes | | | | | | yes |
| massiliensis | | | | | | | | | |
| Bacteroides | −0.06 | | yes | | | | | | yes |
| plebeius | | | | | | | | | |
| Bacteroides sp. | −0.03 | | yes | | | | | | yes |
| 35AE37 | | | | | | | | | |
| Bacteroides | −0.02 | | yes | | yes | | | | yes |
| thetaiotaomicron | | | | | | | | | |
| Bacteroides | −0.02 | | yes | | yes | | | | yes |
| xylanisolvens | | | | | | | | | |
| Lactobacillus | −0.24 | yes | | | yes | yes | | | |
| rhamnosus | | | | | | | | | |
| Lactococcus lactis | −0.01 | yes | | | yes | yes | | | |

TABLE B
Specific Example of Co-occurrence probability of Genus
in samples from antibiotic non-consumers.

| Probability of co- | | |
|---|---|---|
| occurrence | Taxon 1 | Taxon 2 |
| 1 | Anaerostipes | Bacteroides |
| 1 | Anaerostipes | Blautia |
| 1 | Anaerostipes | Clostridium |
| 1 | Anaerostipes | Dorea |
| 1 | Anaerostipes | Faecalibacterium |
| 1 | Anaerostipes | Flavonifractor |
| 1 | Anaerostipes | Pseudobutyrivibrio |
| 1 | Anaerostipes | Roseburia |
| 1 | Bacteroides | Blautia |
| 1 | Bacteroides | Clostridium |
| 1 | Bacteroides | Dorea |
| 1 | Bacteroides | Faecalibacterium |
| 1 | Bacteroides | Flavonifractor |
| 1 | Bacteroides | Pseudobutyrivibrio |
| 1 | Bacteroides | Roseburia |
| 1 | Blautia | Clostridium |
| 1 | Blautia | Dorea |
| 1 | Blautia | Faecalibacterium |
| 1 | Blautia | Flavonifractor |
| 1 | Blautia | Pseudobutyrivibrio |
| 1 | Blautia | Roseburia |
| 1 | Clostridium | Dorea |
| 1 | Clostridium | Faecalibacterium |
| 1 | Clostridium | Flavonifractor |
| 1 | Clostridium | Pseudobutyrivibrio |
| 1 | Clostridium | Roseburia |
| 1 | Dorea | Faecalibacterium |
| 1 | Dorea | Flavonifractor |
| 1 | Dorea | Pseudobutyrivibrio |
| 1 | Dorea | Roseburia |
| 1 | Faecalibacterium | Flavonifractor |
| 1 | Faecalibacterium | Pseudobutyrivibrio |
| 1 | Faecalibacterium | Roseburia |
| 1 | Flavonifractor | Pseudobutyrivibrio |
| 1 | Flavonifractor | Roseburia |
| 1 | Pseudobutyrivibrio | Roseburia |
| 0.99 | Anaerostipes | Collinsella |
| 0.99 | Anaerostipes | Erysipelatoclostridium |
| 0.99 | Anaerostipes | Sarcina |
| 0.99 | Bacteroides | Collinsella |
| 0.99 | Bacteroides | Erysipelatoclostridium |
| 0.99 | Bacteroides | Sarcina |
| 0.99 | Blautia | Collinsella |
| 0.99 | Blautia | Ersipelatoclostridium |
| 0.99 | Blautia | Sarcina |
| 0.99 | Clostridium | Collinsella |
| 0.99 | Clostridium | Ersipelatoclostridium |
| 0.99 | Clostridium | Sarcina |
| 0.99 | Collinsella | Dorea |
| 0.99 | Collinsella | Faecalibacterium |
| 0.99 | Collinsella | Flavonifractor |
| 0.99 | Coilinseila | Pseudobutyrivibrio |
| 0.99 | Collinsella | Roseburia |
| 0.99 | Dorea | Erysipelatoclostridium |
| 0.99 | Dorea | Sarcina |
| 0.99 | Erysipelatoclostridium | Faecalibacterium |
| 0.99 | Erysipelatoclostridium | Flavonifractor |
| 0.99 | Erysipelatoclostridium | Pseudobutyrivibrio |
| 0.99 | Erysipelatoclostridium | Roseburia |
| 0.99 | Faecalibacterium | Sarcina |
| 0.99 | Flavonifractor | Sarcina |
| 0.99 | Pseudobutyrivibrio | Sarcina |
| 0.99 | Roseburia | Sarcina |
| 0.98 | Anaerostipes | Fusicatenibacter |
| 0.98 | Anaerostipes | Intestinibacter |
| 0.98 | Anaerostipes | Parabacteroides |
| 0.98 | Anaerostipes | Subdoligranulum |
| 0.98 | Bacteroides | Fusicatenibacter |
| 0.98 | Bacteroides | Intestinibacter |
| 0.98 | Bacteroides | Parabacteroides |
| 0.98 | Bacteroides | Subdoligranulum |
| 0.98 | Blautia | Fusicatenibacter |
| 0.98 | Blautia | Intestinibacter |
| 0.98 | Blautia | Parabacteroides |
| 0.98 | Blautia | Subdoligranulum |
| 0.98 | Clostridium | Fusicatenibacter |
| 0.98 | Clostridium | Intestinibacter |
| 0.98 | Clostridium | Parabacteroides |
| 0.98 | Clostridium | Subdoligranulum |
| 0.98 | Collinsella | Erysipelatoclostridium |
| 0.98 | Collinsella | Sarcina |
| 0.98 | Dorea | Fusicatenibacter |
| 0.98 | Dorea | Intestinibacter |
| 0.98 | Dorea | Parabacteroides |
| 0.98 | Dorea | Subdoligranulum |
| 0.98 | Erysipelatoclostridium | Sarcina |
| 0.98 | Faecalibacterium | Fusicatenibacter |
| 0.98 | Faecalibacterium | Intestinibacter |
| 0.98 | Faecalibacterium | Parabacteroides |
| 0.98 | Faecalibacterium | Subdoligranulum |
| 0.98 | Flavonifractor | Fusicatenibacter |
| 0.98 | Flavonifractor | Intestinibacter |
| 0.98 | Flavonifractor | Parabacteroides |
| 0.98 | Flavonifractor | Subdoligranulum |
| 0.98 | Fusicatenibacter | Pseudobutyrivibrio |
| 0.98 | Fusicatenibacter | Roseburia |
| 0.98 | Intestinibacter | Pseudobutyrivibrio |
| 0.98 | Intestinibacter | Roseburia |
| 0.98 | Parabacteroides | Pseudobutyrivibrio |
| 0.98 | Parabacteroides | Roseburia |
| 0.98 | Pseudobutyrivibrio | Subdoligranulum |
| 0.98 | Roseburia | Subdoligranulum |
| 0.97 | Anaerostipes | Anaerotruncus |
| 0.97 | Anaerostipes | Lachnospira |
| 0.97 | Anaerotruncus | Bacteroides |
| 0.97 | Anaerotruncus | Blautia |
| 0.97 | Anaerotruncus | Clostridium |
| 0.97 | Anaerotruncus | Dorea |
| 0.97 | Anaerotruncus | Faecalibacterium |
| 0.97 | Anaerotruncus | Flavonifractor |
| 0.97 | Anaerotruncus | Pseudobutyrivibrio |
| 0.97 | Anaerotruncus | Roseburia |
| 0.97 | Bacteroides | Lachnospira |
| 0.97 | Blautia | Lachnospira |
| 0.97 | Clostridium | Lachnospira |
| 0.97 | Collinsella | Fusicatenibacter |
| 0.97 | Collinsella | Intestinibacter |
| 0.97 | Collinsella | Parabacteroides |
| 0.97 | Collinsella | Subdoligranulum |
| 0.97 | Dorca | Lachnospira |
| 0.97 | Erysipelatoclostridium | Fusicatenibacter |
| 0.97 | Erysipelatoclostridium | Intestinibacter |
| 0.97 | Erysipelatoclostridium | Parabacteroides |
| 0.97 | Erysipelatoclostridium | Subdoligranulum |
| 0.97 | Faecalibacterium | Lachnospira |
| 0.97 | Flavonifractor | Lachnospira |
| 0.97 | Fusicatenibacter | Sarcina |
| 0.97 | Intestinibacter | Sarcina |
| 0.97 | Lachnospira | Pseudobutyrivibrio |
| 0.97 | Lachnospira | Roseburia |
| 0.97 | Parabacteroides | Sarcina |
| 0.96 | Sarcina | Subdoligranulum |
| 0.96 | Anaerotruncus | Collinsella |
| 0.96 | Anaerotruncus | Erysipelatoclostridium |
| 0.96 | Anaerotruncus | Sarcina |
| 0.96 | Collinsella | Lachnospira |
| 0.96 | Erysipelatoclostridium | Lachnospira |
| 0.96 | Fusicatenibacter | Intestinibacter |
| 0.96 | Fusicatenibacter | Parabacteroides |
| 0.96 | Fusicatenibacter | Subdoligranulum |
| 0.96 | Intestinibacter | Parabacteroides |
| 0.96 | Intestinibacter | Subdoligranulum |
| 0.96 | Lachnospira | Sarcina |
| 0.951 | Parabacteroides | Subdoligranulum |
| 0.951 | Anaerotruncus | Fusicatenibacter |
| 0.951 | Anaerotruncus | Intestinibacter |
| 0.951 | Anaerotruncus | Parabacteroides |
| 0.951 | Anaerotruncus | Subdoligranulum |
| 0.951 | Fusicatenibacter | Lachnospira |
| 0.951 | Intestinibacter | Lachnospira |
| 0.951 | Lachnospira | Parabacteroides |
| 0.951 | Lachnospira | Subdoligranulum |
| 0.95 | Alistipes | Anaerostipes |
| 0.95 | Alistipes | Bacteroides |
| 0.95 | Alistipes | Blautia |
| 0.95 | Alistipes | Clostridium |
| 0.95 | Alistipes | Dorea |
| 0.95 | Alistipes | Faecalibacterium |
| 0.95 | Alistipes | Flavonifractor |
| 0.95 | Alistipes | Pseudobutyrivibrio |
| 0.95 | Alistipes | Roseburia |
| 0.95 | Anaerostipes | Intestinimonas |
| 0.95 | Bacteroides | Intestinimonas |
| 0.95 | Blautia | Intestinimonas |
| 0.95 | Clostridium | Intestinimonas |
| 0.95 | Dorea | Intestinimonas |
| 0.95 | Faecalibacterium | Intestinimonas |
| 0.95 | Flavonifractor | Intestinimonas |
| 0.95 | Intestinimonas | Pseudobutyrivibrio |
| 0.95 | Intestinimonas | Roseburia |
| 0.941 | Anaerotruncus | Lachnospira |
| 0.94 | Alistipes | Collinsella |
| 0.94 | Alistipes | Erysipelatoclostridium |
| 0.94 | Alistipes | Sarcina |
| 0.94 | Collinsella | Intestinimonas |
| 0.94 | Erysipelatoclostridium | Intestinimonas |
| 0.94 | Intestinimonas | Sarcina |
| 0.931 | Alistipes | Fusicatenibacter |
| 0.931 | Alistipes | Intestinibacter |
| 0.931 | Alistipes | Parabacteroides |
| 0.931 | Alistipes | Subdoligranulum |
| 0.931 | Fusicatenibacter | Intestinimonas |
| 0.931 | Intestinibacter | Intestinimonas |
| 0.931 | Intestinimonas | Parabacteroides |
| 0.931 | Intestinimonas | Subdoligranulum |
| 0.93 | Anaerostipes | Streptococcus |
| 0.93 | Bacteroides | Streptococcus |
| 0.93 | Blautia | Streptococcus |
| 0.93 | Clostridium | Streptococcus |
| 0.93 | Dorea | Streptococcus |
| 0.93 | Faecalibacterium | Streptococcus |
| 0.93 | Flavonifractor | Streptococcus |
| 0.93 | Pseudobutyrivibrio | Streptococcus |
| 0.93 | Roseburia | Streptococcus |
| 0.922 | Alistipes | Anaerotruncus |
| 0.922 | Alistipes | Lachnospira |
| 0.922 | Anaerotruncus | Intestinimonas |
| 0.922 | Intestinimonas | Lachnospira |
| 0.921 | Collinsella | Streptococcus |
| 0.921 | Erysipelatoclostridium | Streptococcus |
| 0.921 | Sarcina | Streptococcus |
| 0.911 | Fusicatenibacter | Streptococcus |
| 0.911 | Intestinibacter | Streptococcus |
| 0.911 | Parabacteroides | Streptococcus |
| 0.911 | Streptococcus | Subdoligranulum |
| 0.91 | Anaerostipes | Oscillibacter |
| 0.91 | Bacteroides | Oscillibacter |
| 0.91 | Blautia | Oscillibacter |
| 0.91 | Clostridium | Oscillibacter |
| 0.91 | Dorea | Oscillibacter |
| 0.91 | Faecalibacterium | Oscillibacter |
| 0.91 | Flavonifractor | Oscillibacter |
| 0.91 | Oscillibacter | Pseudobutyrivibrio |
| 0.91 | Oscillibacter | Roseburia |
| 0.902 | Alistipes | Intestinimonas |
| 0.902 | Anaerotruncus | Streptococcus |
| 0.902 | Lachnospira | Streptococcus |
| 0.901 | Collinsella | Oscillibacter |
| 0.901 | Erysipelatoclostridium | Oscillibacter |
| 0.901 | Oscillibacter | Sarcina |
| 0.892 | Fusicatenibacter | Oscillibacter |
| 0.892 | Intestinibacter | Oscillibacter |
| 0.892 | Oscillibacter | Parabacteroides |
| 0.892 | Oscillibacter | Subdoligranulum |
| 0.89 | Anaerostipes | Marvinbryantia |
| 0.89 | Bacteroides | Marvinbryantia |
| 0.89 | Blautia | Marvinbryantia |
| 0.89 | Clostridium | Marvinbryantia |
| 0.89 | Dorea | Marvinbryantia |
| 0.89 | Faecalibacterium | Marvinbryantia |
| 0.89 | Flavonifractor | Marvin bryantia |
| 0.89 | Marvinbryantia | Pseudobutyrivibrio |
| 0.89 | Marvinbryantia | Roseburia |
| 0.884 | Al isti pes | Streptococcus |
| 0.884 | Intestinimonas | Streptococcus |
| 0.883 | Anaerotruncus | Oscillibacter |
| 0.883 | Lachnospira | Oscillibacter |
| 0.881 | Collinsella | Marvinbryantia |
| 0.881 | Erysipelatoclostridium | Marvinbryantia |
| 0.881 | Marvinbryantia | Sarcina |
| 0.872 | Fusicatenibacter | Marvin bryantia |
| 0.872 | Intestinibacter | Marvinbryantia |
| 0.872 | Marvinbryantia | Parabacteroides |
| 0.872 | Marvinbryantia | Subdoligranulum |
| 0.87 | Anaerostipes | Bilophila |
| 0.87 | Bacteroides | Bilophila |
| 0.87 | Bilophila | Blautia |
| 0.87 | Bilophila | Clostridium |
| 0.87 | Bilophila | Dorea |
| 0.87 | Bilophila | Faecalibacterium |
| 0.87 | Bilophila | Flavonifractor |
| 0.87 | Bilophila | Pseudobutyrivibrio |
| 0.87 | Bilophila | Roseburia |
| 0.864 | Alistipes | Oscillibacter |
| 0.864 | Intestinimonas | Oscillibacter |
| 0.863 | Anaerotruncus | Marvinbryantia |
| 0.863 | Lachnospira | Marvinbryantia |
| 0.861 | Bilophila | Collinsella |
| 0.861 | Bilophila | Erysipelatoclostridium |
| 0.861 | Bilophila | Sarcina |
| 0.853 | Bilophila | Fusicatenibacter |
| 0.853 | Bilophila | Intestinibacter |
| 0.853 | Bilophila | Parabacteroides |
| 0.853 | Bilophila | Subdoligranulum |

In a further aspect of the present disclosure, a platform for determining inhibitors of bacterial metabolites.

The concept of “drugging the microbiome” has emerged as a therapeutic approach to avoid targeting human cells directly, by targeting receptors and enzymes belonging to microbiota. This concept can be especially applied to inhibit microbial enzymes that produce metabolites with adverse effects in the human body. This new approach also aims at evading to knock-down human enzymes function by gene therapy methods.

One of the most reported cases is the production of TMA mediated by human microbiota from dietary choline and L-carnitine, through the action of CutC/D and CntA/CntB enzymes. TMA is a precursor of trimethylamine N-oxide (TMAO); metabolite that has been related with a high risk of cardiovascular and renal diseases, and additionally, high levels of TMAO appear to trigger atherosclerosis in mice. Recently, inhibitors for the TMA-producing enzymes have been suggested.

Embodiments of a method can include a new pipeline to identify and target enzymes in is bacteria is proposed. Embodiments can include associated therapeutic compositions.

Embodiments of a method can include bacterial proteins that produce specific detrimental metabolites. Embodiments can include using identified bacterial proteins as targets to design new small molecules inhibitors. Embodiments can include therapeutic compositions including the one or more small molecule inhibitors.

Embodiments can include identifying new enzymes that produce detrimental metabolites, and/or determining and/or generating one or more new drugs to inhibit those enzymes. Embodiments can include the new drugs (e.g., in any suitable therapeutic composition form; etc.). Embodiments can include one or more new drugs and/or suitable therapeutic compositions that can be used to prevent the production of detrimental metabolites by bacteria, helping with the treatment of one or more of several conditions or diseases.

Embodiments (e.g., embodiments of a method such as including a pipeline described herein; etc.) can function to, include, and/or otherwise be associated with finding orthologous metabolites producing enzymes to those already known, such as by sequence matching against reference proteomes and/or other sources such as NCBI and/or any suitable databases and/or sources.

Specific Examples:

In specific examples, to this end, several alignment algorithms can be used (e.g., one or more of BLAST, FASTA, CLUSTAL, among others, etc.). A sequence similarity network can be built to obtain a representative sequence for each taxonomic order (e.g., phylum), such as with any suitable approach described in U.S. application Ser. No. 16/103,830 filed 14, Aug. 2018, and to identify every protein family involved in the production of such metabolites. Additionally or alternatively, one or more metabolism predictor tools can be used to identify one or more metabolic pathways for metabolites bacterial production, such as any suitable metabolism-associated tools and/or approaches described in PCT Application PCT/US19/22807 filed 18, Mar. 2019.

Once representative sequences for metabolites enzymes producers have been identified (and/or at any suitable time and/or frequency), a structural model of those enzymes can be either obtained from the Protein Data Bank (PDB) and/or by homology modelling and/or by any suitable databases and/or approaches. The active site of those enzymes can be identified either by tools that allow pocket prediction, and/or by analogues structures in the PDB or by literature information about the binding site, and/or by a known molecule whose better placement into the structure can be predicted, and/or by any suitable approach.

Once the active site has been identified (and/or at any suitable time and frequency), competitive inhibitors can be obtained. Competitive inhibition is a type of enzyme inhibition, where binding at the active site of the enzyme prevents the binding of its substrate and vice versa. In other words, the substrate and the inhibitor cannot bind the active site at the same time.

Thus, new possible inhibitors can be found, such as by virtual high throughput screening using molecular docking (and/or other suitable approaches) on a big library of compounds (as an example, CHEMBL, CHEMSPIDER, ZINC, etc.) using the enzyme structure and the active site obtained as a target. The best candidates can be defined as those with the best docking binding energies; but any suitable ranking of candidates can be applied In specific 30 examples, Candidates can be filtered by a druggability assessment, for example, by obtaining Lipinski's rules: Those rules include: molecular weight <500 daltons, number of H-bonds donor <5, number of H-bonds acceptor <10, number of N and 0 atoms <15, range of partition coefficient logP between −2 and 5, number of rotatable bonds <10, ring number <10, Only candidates that pass this filter will be considered. Additionally, molecules that do not pass the Lipinski rules can be modified by in-silico tools (as an example, fragment-based design, phartnacophore-based design) to obtain candidates with better druggable properties. However, any suitable conditions can be applied for filtering.

Some examples of metabolites whose production can be inhibited by the implementation of this pipeline can include: industrial chemicals and pollutants, dietary compounds and pharmaceuticals, and/or other suitable metabolites. For example, bacterial beta-glucuronidase enzymes are sometimes responsible of detrimental metabolism on drugs used for several diseases. Sonic of these drugs are cutTently used to treat from a simple inflammation (ketoprofen, diclofenac) until cancer. Beta glucuronidase enzymes can provoke that those drugs become into toxic metabolites. To design drugs as inhibitors for this class of enzymes can be useful to generate “companion drugs”, to be used at the same time with the altered drugs. As an example, one well reported drug that it is altered by these enzymes is called Irinotecan. This anti-cancer drug is converted into a new compound by those enzymes, provoking diarrhea in patients, among other secondary effects.

Additionally, the identification of some enzymes inhibitors can help to reduce the overproduction of some compounds in diseases such as chronic kidney disease, such as phenol and indoles. Some inhibitors can also aim to reduce overproduction of acetaldehyde mediated by bacterial alcohol dehydrogenase. The excessive accumulation of acetaldehyde can lead to some diseases such as colorectal cancer.

Embodiments can include, based on the implementation of approaches described herein (e.g., pipeline described herein), new drugs as inhibitors of bacterial metabolites production;

and/or can include obtainment of the new drugs, such as based on the data.

Embodiments can include a method to identify new bacterial proteins involved in the production of undesired metabolites.

Embodiments can include a method to identify and generate new inhibitors of bacterial proteins involved in the production of undesired metabolites.

Embodiments can include one or more therapeutic compositions including such bacterial proteins and/or inhibitors.

Embodiments of the method can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microhiome compositions and/or functional features of subjects.

Embodiments of the method and/or system can include every combination and permutation of the various system components and the various method processes, including any variants (e.g., embodiments, variations, examples, specific examples, figures, etc.), where portions of embodiments of the method and/or processes described herein can be performed. asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method and/or system can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMS, ROMs, flash memory, EEPROMs, optical devices (CD or DVD), hard drives, floppy drives, or any suitable device.

The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method, system, and/or variants without departing from the scope defined in the claims.

What is claimed is:

1. A method for treating a microorganism-related condition in a patient, the method comprising:
detecting microorganisms in a set of samples collected from a population;
comparing a relative abundance of and co-occurrence between different microbial taxa in the set of samples;
associating a change in the relative abundance of or the co-occurrence between the microbial taxa with samples from people, among the population, with the microorganism-related condition and samples from people, among the population, without the microorganism-related condition to determine a target taxa;
identifying a blend of bacteriophages, the blend being configured to remove the target taxa from a community of microorganisms; and
administering a therapeutic composition comprising the blend to the patient with the microorganism-related condition.

2. The method of claim 1, wherein the target taxa comprises a taxon directly correlated with an occurrence of the microorganism-related condition among the population.

3. The method of claim 1, wherein the target taxa comprises a taxon co-occurring with a taxon directly correlated with an occurrence of the microorganism-related condition among the population.

4. A method for treating a microorganism-related condition in a patient, the method comprising:
detecting microorganisms in a set of samples collected from a population;
comparing a relative abundance of and co-occurrence between different microbial taxa in the set of samples;
associating a change in the relative abundance of or the co-occurrence between the microbial taxa with samples from people, among the population, with the microorganism-related condition and samples from people, among the population, without the microorganism-related condition to determine a target taxa;
identifying a blend of therapeutic microorganisms, the blend being configured to change an abundance of the target taxa in a community of microorganisms; and
administering a therapeutic composition comprising the blend to the patient with the microorganism-related condition.

5. The method of claim 4, wherein the target taxa comprises a taxon directly correlated with an occurrence of the microorganism-related condition among the population.

6. The method of claim 4, wherein the target taxa comprises a taxon co-occurring with a taxon directly correlated with an occurrence of the microorganism-related condition among the population.

7. The method of claim 4, wherein the blend is configured to up-regulate the abundance of the target taxa by directly repopulating the target taxa.

8. The method of claim 4, wherein the blend is configured to up-regulate the abundance of the target taxa by repopulating one or more taxa haying a high probability of co-occurrence with the target taxa.

9. The method of claim 4, wherein the blend comprises a strain or species selected from the group consisting of Enterococcus faecium, Lactobacillus rhamnosus, Lactobacillus salivarius, Bifidobacterium adolescentis, Bifidobacterium animalis, Lactobacillus gasseri, Bifidobacterium breve, Bifidobacterium catenulatum, Bifidobacterium pseudocatenulatum, Bifidobacterium stercoris, Lactobacillus reuteri, Lactobacillus fermentutn, Pediococcus pentosaceus, Lactobacillus helveticus, Lactobacillus brevis, Lactococcus lacus, Bacteroides xylanisolvens.

10. The method of claim 4, wherein the blend comprises a strain or species selected from the group consisting of: Faecalibacterium prausnitzii, Roseburia faecis, Roseburia hominis, Roseburia intestinalis, Anaerostipes caccae, Anaerostipes rhamnosivorans, Eubacterium limosum, Eubacterium sp. ARC. 2, Subdoligranulum variabde, Akkermansia muciniphila, Bifidobacterium adolescentis, Bifidobacterium animalis, Bifidobacterium breve, Bifidobacterium catenulatum, Bifidobacterium crudilactis, Bifidobacterium dentium, Bifidobacterium pseudocatenulatum, Bifidobacterium stercoris, Bifidobacterium thermacidophilum, Methanobrevibacter smithii, Roseburia sp. 499, Bacteroides dorei, Bacteroides massiliensis, Bacteroides plebeius, Bacteroides sp. 35AE37, Bacteroides thetaiotaomicron, Bacteroides xylanisolvens, Lactobacillus rhamnosus, Lactococcus lactis, Enterococcus faecium, Lactobacillus salivarius, Lactobacillus gasseri, Lactobacillus reuteri, Lactobacillus fermentum, Pediococcus pentosaceus, Lactobacillus helveticus, Lactobacillus brevis.

11. A method for identifying new bacteria-produced antibacterial compounds, the method comprising:
generating a database of antibacterial compounds produced by bacteria by screening known antibacterial compounds-producing microorganisms and antibacterial compounds;
identifying, by a processor, binding regions of the antibacterial compounds from the database that bind other microorganisms by comparing sequence alignment of curated antibacterial compounds with a sequence alignment of reference proteomes; and
identify new bacteria-produced antibacterial compounds based on the identified peptide motifs.

12. The method of claim the curated antibacterial compounds include lantibiotics, bateriocins, and microcin.

13. The method of claim 11, wherein reference proteomes are selected from Uniprot database or NCBI database.

14. The method of claim 11, wherein comparing sequence alignment is performed using a sequence alignment algorithm selected from the group comprising BLAST, FASTA, and Clustal.

15. The method of claim 11, further comprising: analyzing a structure of peptide motifs to determine a set of peptide motifs among the identified peptide motifs which can interact with proteins from microrganisms; and
for the set of peptide motifs, modeling an interaction between each peptide motifs with known targets from microorganism that are inhibited by an action of a known antibacterial peptide.

16. A method of producing a therapeutic composition, the method comprising:
identifying a protein from bacteria that produce metabolites underlying a microorganism-related condition;
identifying, by a processor, a first inhibitor for the identified protein and a second inhibitor of a protein orthologous to the identified protein using virtual high-throughput screening; and
producing a therapeutic composition comprising one or both of the first inhibitor and the second inhibitor.
